(19) United States
(12) Patent Application Publication
(10) Pub. No.: US 2007/0073152 A1
(43) Pub. Date: Mar. 29, 2007

(54) Systems and methods for acquiring images simultaneously

(75) Inventor: Michael Joseph Washburn, Brookfield, WI (US)

Correspondence Address: PATRICK W. RASCHE (12553 - 1000), ARMSTRONG TEASDALE LLP, ONE METROPOLITAN SQUARE, SUITE 2600, ST. LOUIS, MO, 63102-2740, US

(73) Assignee: General Electric Company

(21) Appl. No.: 11/225,552

(22) Filed: Sep. 13, 2005

(51) Int. Cl. A61B 8/00

(52) U.S. Cl. 600/441; 600/437

(57) ABSTRACT

A method for acquiring images simultaneously is described. The method includes simultaneously acquiring a first image with a second image, where the first image includes at least one of a spatially compounded image and a spatially non-compounded image, and the second image includes one of an M mode image and a Doppler image.

### CROSS REFERENCE TO RELATED APPLICATIONS

This application is related to co-pending U.S. patent application having Ser. No. 11/138,199, titled “Methods and Systems For Acquiring Ultrasound Image Data”, and filed on May 26, 2005.

### BACKGROUND OF THE INVENTION

This invention relates generally to medical imaging systems and more particularly to systems and methods for acquiring images simultaneously.

Premium medical diagnostic ultrasound imaging systems require a comprehensive set of imaging modes. These are the major imaging modes used in clinical diagnosis and include spectral Doppler, color flow, B mode and M mode. The color flow mode creates a color flow image, the B mode creates a B mode image, the Doppler mode creates a Doppler image, and the M mode creates an M mode image. In the B mode, such ultrasound imaging systems create two-dimensional images of tissue in which the brightness of a pixel is based on the intensity of an echo return. Alternatively, in a color flow imaging mode, a movement of fluid (e.g., blood) or alternatively a tissue can be imaged. Measurement of blood flow in a heart and a plurality of vessels by using Doppler effect is well known. A phase shift of backscattered ultrasound waves may be used to measure a velocity of the backscatterers from tissue or alternatively blood. A Doppler shift may be displayed using different colors to represent speed and direction of flow. In the spectral Doppler imaging mode, a power spectrum of a plurality of Doppler frequency shifts are computed for visual display as velocity-time waveforms.

However, each of the Doppler, color flow, M mode, and the B mode image, when displayed, are limited in their ability to provide information regarding an anatomy. For example, when the Doppler image is displayed on a display screen, the Doppler image provides physiological information regarding the anatomy without providing a structure of the anatomy. As another example, when the B mode is displayed on a display screen, the B mode image provides the structure without providing the physiological information.

### BRIEF DESCRIPTION OF THE INVENTION

In one aspect, a method for acquiring images simultaneously is described. The method includes simultaneously acquiring a first image with a second image, where the first image includes at least one of a spatially compounded image and a spatially non-compounded image, and the second image includes one of an M mode image and a Doppler image.

In another aspect, a processor is described. The processor is configured to control a simultaneous acquisition of a first image with a second image, where the first image includes at least one of a spatially compounded image and a spatially non-compounded image, and the second image includes one of an M mode image and a Doppler image.

In yet another aspect, an ultrasound imaging system is described. The ultrasound imaging system includes a plurality of transducer elements configured to receive a plurality of ultrasound echoes and convert the ultrasound echoes to a plurality of electrical signals, a beamformer board coupled to the transducer elements and configured to generate a receive beam from the electrical signals, and a first image processor coupled to the beamformer and configured to generate a first image output from the receive beam. The ultrasound imaging system further includes a second image processor coupled to the beamformer and configured to generate a second image output from the receive beam. The ultrasound imaging system includes a master processor configured to control the transducer elements, the beamformer, the first image processor, and the second image processor to simultaneously acquire a first image formed from the first image output with a second image formed from the second image output, where the first image includes at least one of a spatially compounded image and a spatially non-compounded image, and the second image includes one of an M mode image and a Doppler image.

### BRIEF DESCRIPTION OF THE DRAWINGS

FIG. 1 is a block diagram of an embodiment of an ultrasound imaging system implementing systems and methods for acquiring images simultaneously.

FIG. 2 illustrates an embodiment of an acquisition of an image of an object by using the ultrasound imaging system of FIG. 1.

FIG. 3 illustrates an embodiment of different regions of a spatially compounded frame generated by using the ultrasound imaging system of FIG. 1.

FIG. 4 illustrates a block diagram of an embodiment of an acquisition system that is used in connection with the ultrasound imaging system of FIG. 1.

FIG. 5 is an embodiment of method for acquiring a sequence of frames in real time by using the ultrasound imaging system of FIG. 1.

FIG. 6 is an embodiment of a method for acquiring images simultaneously.

FIG. 7 is an alternative embodiment of a method for acquiring images simultaneously.

FIG. 8 is yet another embodiment of a method for acquiring images simultaneously.

### DETAILED DESCRIPTION OF THE INVENTION

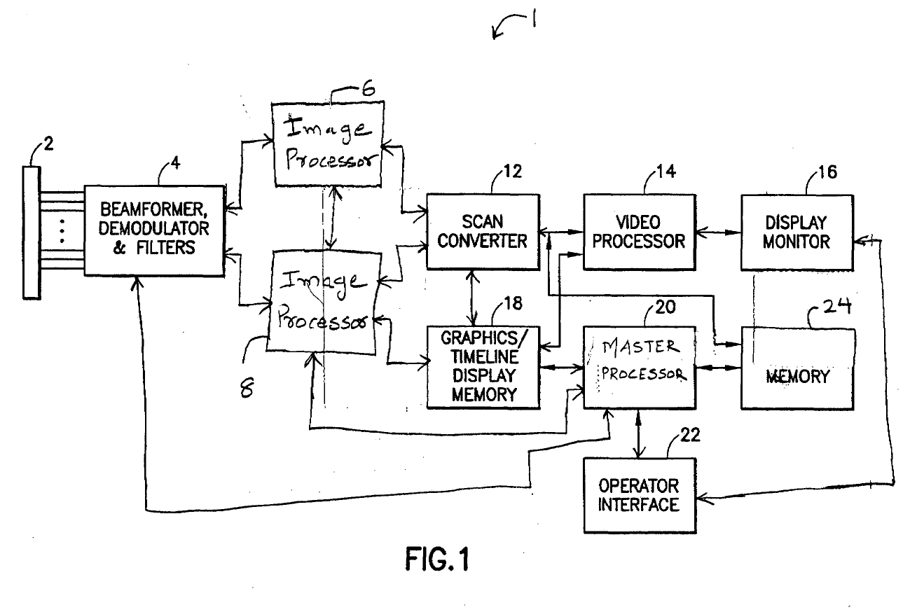

FIG. 1 is an embodiment of a block diagram of an embodiment of an ultrasound imaging system 1 implementing systems and methods for acquiring images simultaneously. Ultrasound imaging system 1 includes a transducer 2, a beamformer board 4, an image processor 6, an image processor 8, a scan converter 12, a video processor 14, a display monitor 16, a graphics/timeline display memory 18, a master processor 20, an operator interface 22, and a cine memory 24. Image processor 6 is a B mode processor. In an alternative embodiment, image processor 6 is a color flow processor. In yet another alternative embodiment, the color flow processor is connected in parallel with the B mode processor. In an alternative embodiment, image processor 6 performs spatial compounding. Examples of image processor 8 include an M mode processor and a Doppler processor. Examples of each of memory 24 and graphics/timeline display memory 18 include a hard disk, a compact disc—read only memory (CD-ROM), a magneto-optical disk (MOD), and a digital versatile disc (DVD). Display monitor 16 may be a cathode ray tube (CRT) or alternatively a liquid crystal device (LCD). Examples of operator interface 22 include a mouse, a keyboard, a trackball, a touch sensitive screen, and a control panel. A processor, such as image processor 6, image processor 8, video processor 14, master processor 20, is not limited to just those integrated circuits referred to in the art as a processor, but broadly refers to a computer, a microcontroller, a microcomputer, a programmable logic controller, an application specific integrated circuit, and other programmable circuits.

A main data path begins with a plurality of analog radio frequency (RF) signals to the beamformer board 4 from the transducer 2. The beamformer board 4 is responsible for transmit and receive beamforming. A plurality of signal inputs to the beamformer board 4 are the analog RF signals from a plurality of transducer elements, such as piezoelectric crystals, within transducer 2. The beamformer board 4, which includes a beamformer, a demodulator and a plurality of finite impulse response (FIR) filters, outputs two summed digital baseband I and Q receive beams formed from the analog RF signals. The analog RF signals are derived from reflected ultrasound signals generated from respective focal zones of a plurality of transmitted ultrasound pulses. The I and Q receive beams are sent to the FIR filters, which are programmed with filter coefficients to pass a band of frequencies centered at a fundamental frequency or alternatively at a subharmonic frequency. In an alternative embodiment, the beamformer board 4 may not include the demodulator and the FIR filters.

Data output from the filters is sent to a midprocessor subsystem, where it is processed according to an acquisition mode and output as processed vector data including B mode intensity data, M mode data, Doppler data, and color flow data. The midprocessor subsystem includes image processors 6 and 8. The B mode processor converts the I and Q receive beams having a signal envelope and received from beamformer board 4 into a log-compressed version of the signal envelope. The B mode processor images a time-varying amplitude of the signal envelope as a gray scale. The signal envelope is a magnitude of a vector which I and Q represent. The magnitude of the vector is a square root of a sum of squares of I and Q. The B mode intensity data is output from the B mode processor to the scan converter 12.

The scan converter 12 accepts the B mode intensity data, interpolates where necessary, and converts the B mode intensity data into X-Y format for video display. Scan converted frames output from scan converter 12 are passed to a video processor 14, which maps the scan converted frames to a gray-scale mapping for video display. Gray-scale image frames output from video processor 14 are sent to the display monitor 16 for display.

A B mode image displayed by display monitor 16 is produced from the gray-scale image frames in which each datum indicates an intensity and/or brightness of a respective pixel on the display monitor 16. One of the gray-scale image frames may include a 256×256 data array in which each intensity datum is an 8-bit binary number that indicates pixel brightness. Each pixel has an intensity value which is a function of a backscatter cross section of a sample volume in response to the transmitted ultrasonic pulses and the gray-scale mapping employed. The B mode image represents a tissue and/or blood flow in a plane through the sample volume of a body being imaged.

The color flow processor is used to provide a real-time two-dimensional color flow image of blood velocity in an imaging plane. A frequency of sound waves reflecting from an inside of the sample volume, such as, blood vessels and heart cavities, is shifted in proportion to the blood velocity of blood cells of the sample volume, positively shifted for cells moving towards the transducer 2 and negatively for those moving away from the transducer 2. The blood velocity is calculated by measuring a phase shift from a transmit firing to another transmit firing at a specific range gate. Instead of measuring a Doppler spectrum at one range gate, mean blood velocity from multiple vector positions and multiple range gates along each vector are calculated, and a two-dimensional image is generated. The color flow processor receives the I and Q receive beams from the beamformer board 4 and processes the beams to calculate the mean blood velocity, a variance representing blood turbulence, and total prenormalization power for the sample volume within an operator-defined region. The color flow processor combines the mean blood velocity, the variance, and the total prenormalization power into two final outputs, one primary and one secondary. The primary output is either the mean blood velocity and/or the prenormalization power. The secondary output is either the variance or the prenormalization power. Which two of the mean blood velocity, the variance, and the total prenormalization power are displayed is determined by a display mode selected by an operator via the operator interface 22. Any two of the mean blood velocity, the variance, and the total prenormalization power are sent to the scan converter 12. The color flow mode displays hundreds of adjacent sample volumes simultaneously, all laid over the B mode image and color-coded to represent each sample volume's velocity.

In any of the B mode, color flow mode, M mode, and Doppler mode, master processor 20 activates transducer 2 to transmit at least one of a series of multi-cycle, such as 4-8 cycles, transmit firings, which are tone bursts focused at the same transmit focal position with the same transmit characteristics. Each transmit firing is an ultrasound pulse. The transmit firings are periodically fired at a pulse repetition frequency (PRF). Alternatively, the transmit firings are filed continuously with lesser time between any two of the transmit firings than a time when the transmit firings are fired periodically. The PRF is typically in a kilo-hertz range. A series of the transmit firings focused at the same transmit focal position are referred to as a “packet”. Each transmit firing propagates through the sample volume being scanned and is reflected as the reflected ultrasound signals by ultrasound scatterers, such as, blood cells, of the sample volume. The reflected ultrasound signals are detected by the transducer elements of the transducer 2 and then formed into the I and Q receive beams by the beamformer 4. The scan converter 12 performs a coordinate transformation of the Doppler data, M mode data, color flow data, and the B mode intensity data from a polar coordinate sector format or alternatively a Cartesian coordinate linear format to scaled Cartesian coordinate display pixel data, which is stored in the scan converter 12.

If an image to be displayed on display monitor 16 is a combination of the B mode image and the color flow image, then both the B mode and the color flow images are passed to the video processor 14, which maps the B mode data to a gray map and maps the color flow data to a color map, for video display. In a displayed image, the color flow image is superimposed on the B mode intensity data.

Successive frames of the color flow and/or B mode data are stored in a memory 24 on a first-in, first-out basis. The memory 24 is like a circular image buffer that runs in the background, capturing data that is displayed in real time to the operator. When the operator freezes a displayed image by operation of the operator interface 22, the operator has the capability to view data previously captured in memory 24.

The Doppler processor integrates and/or sums, over a specific time interval, and samples the I and Q receive beams. The integration interval and lengths of the transmit firings together define a length of the sample volume as specified by the operator. The I and Q receive beams pass through a wall filter which rejects any clutter in the beams corresponding to stationary or alternatively very slow-moving tissue to generate a filtered output. The filtered output is fed into a spectrum analyzer, which typically takes Fast Fourier Transforms (FFTs) over a moving time window of 32 to 128 samples to generate FFT power spectrums. Each FFT power spectrum is compressed by a compressor and then output as the Doppler data by the Doppler processor to the graphics/timeline display memory 18. The video processor 14 maps the Doppler data output from the Doppler processor to a gray scale for display on the display monitor 16 as a single spectral line at a particular time point in a Doppler velocity versus time spectrogram.

For M mode imaging, master processor 20 controls transducer 2 to focus the transmit firings are focused along an ultrasound single scan line. In an alternative embodiment, for M mode imaging, master processor 20 controls transducer 2 to focus each of the transmit firings along a plurality of discrete ultrasound scan lines, either simultaneously or sequentially. The M mode processor includes the B mode processor or alternatively the Doppler processor for generating amplitude, velocity, energy, and/or other information along the ultrasound scan line. An M mode image represents a structure of the sample volume or alternatively a movement of the sample volume along an ultrasound scan line as a function of time. The M mode image represents a depth on one axis and time on another axis.

System control is centered in the master processor 20, which accepts operator inputs through operator interface 22 and in turn controls at least one of transducer 2, beamformer board 4, image processor 6, image processor 8, scan converter 12, video processor 14, display monitor 16, graphics/timeline display memory 18, and memory 24. Master processor 20 accepts inputs from the operator via the operator interface 22 as well as system status changes, such as acquisition mode changes, and makes appropriate changes to at least one of transducer 2, beamformer board 4, image processor 6, image processor 8, scan converter 12, video processor 14, display monitor 16, graphics/timeline display memory 18, and memory 24.

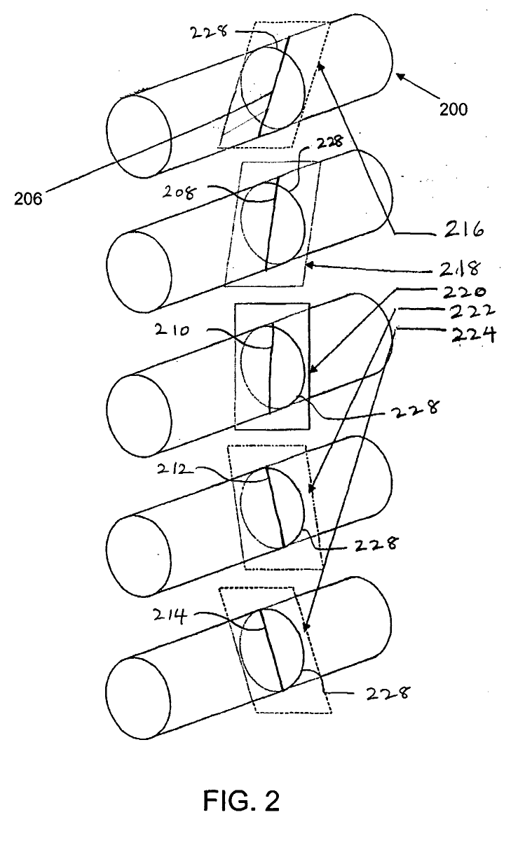

FIG. 2 illustrates an embodiment of an acquisition of an image of an object 200, which is an example of the sample volume. The acquisition is performed using the ultrasound system 1 (FIG. 1). It should be noted that although the image of the object 200 is a volume, different images may be acquired, such as, for example, two-dimensional (2D) images. The image of the object 200 is defined by a plurality of cross-sections 206, 208, 210, 212, and 214 acquired by a plurality of spatially non-compounded frames 216, 218, 220, 222, and 224 to generate an imaged volume.

Image processor 6 performs spatial compounding by combining at least two of frames 216, 218, 220, 222, and 224 of the B mode intensity data from multiple co-planar views of the same sample volume into one spatially compounded frame of data for display. Frames 216, 218, 220, 222, and 224 are acquired in a repeating manner from different lines-of-sight. For example, the same cross-sectional slice 228 of the object 200 is interrogated by the transmit firings from five different directions along frames 216, 218, 220, 222, and 224. As each frame 216, 218, 220, 222, and 224 is acquired, the frame is combined with the previously acquired frames to produce the spatially compounded frame in a geometric space of an un-steered frame.

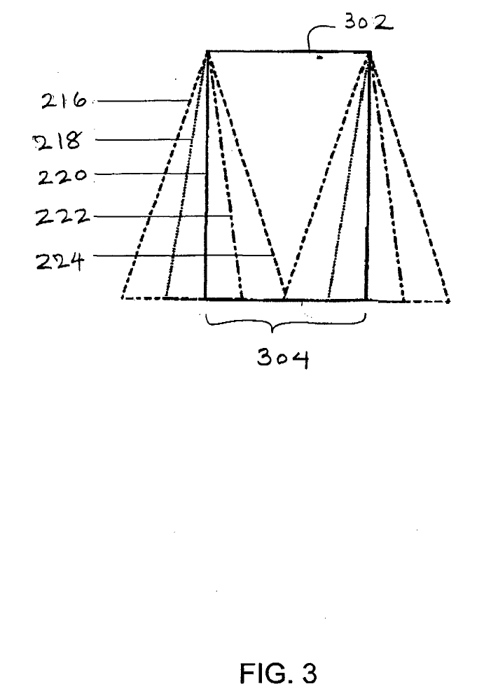

FIG. 3 illustrates an embodiment of different regions of a spatially compounded frame 302 that includes overlapping regions of the frames 216, 218, 220, 222, and 224. As shown, the spatially compounded frame 302 has a geometry of the un-steered frame. In the example, a bottom portion 304 of the spatially compounded frame 302 is formed by combining the B mode intensity data from all five directions along frames 216, 218, 220, 222, and 224. The remainder of the spatially compounded frame 302 is a result of a combination of three or alternatively four frames, depending on a number of frames that overlap a region.

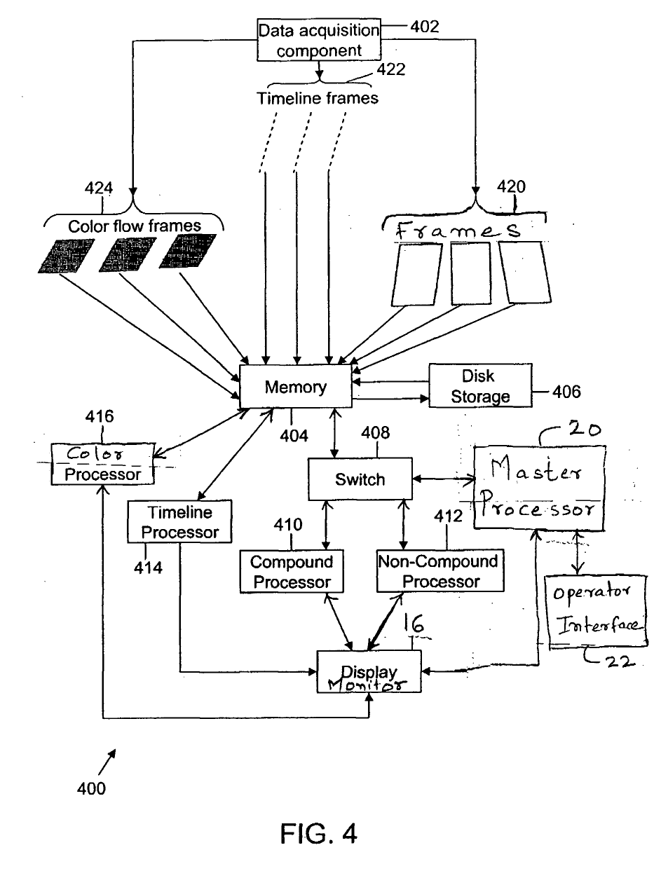

FIG. 4 illustrates a block diagram of an embodiment of an acquisition system 400 that is used in connection with the ultrasound system 1. The acquisition system 400 includes a data acquisition component 402 which includes the transducer 2 and the beamformer board 4. The acquisition system 400 further includes a memory 404, a disk storage 406, a switch 408, a compound processor 410, a non-compound processor 412, a timeline processor 414, a color processor 416, display monitor 16, master processor 20, and operator interface 22. An example of the memory 404 includes a short term memory, such as a random access memory. An example of disk storage 406 includes a long term memory, such as a read only memory. A processor, such as compound processor 410, non-compound processor 412, timeline processor 414, color processor 416, is not limited to just those integrated circuits referred to in the art as a processor, but broadly refers to a computer, a microcontroller, a microcomputer, a programmable logic controller, an application specific integrated circuit, and other programmable circuits.

Master processor 20 controls transducer 2 to convert electrical, such as RF, signals into the transmit firings, such as B mode pulses, M mode pulses, and Doppler pulses. The transmit firings are reflected from the sample volume to generate the reflected ultrasound signals. Master processor 20 controls transducer 2 to receive the reflected ultrasound signals and generates the I and Q receive beams from which frames 420 including the B mode data, timeline frames 422 including one of the Doppler data and the M mode data, and color flow frames 424 including the color flow data are generated. A number of the frames 420, timeline frames 422, and color flow frames 424 are not limited to that shown in FIG. 4. For example, color flow frames 424 may include four frames instead of three.

Frames 420, timeline frames 422, and color flow frames 424 are stored in memory 404. Disk storage 406 is provided for storing desired frames among frames 420, timeline frames 422, and color flow frames 424 for later recall and display. Switch 408 is also provided and is operated by the operator via the operator interface 22. The switch 408 allows the operator to select from frames 420 in memory 404 and/or disk storage 406 to be provided to compound processor 410 and/or non-compound processor 412 to process frames 420. When switch 408 is in a first position, master processor 20 controls compound processor 410 to perform the B mode processing, spatial compounding, scan conversion, and video processing on at least two of frames 420 to generate a spatially compounded image displayed on display monitor 16. When switch 408 is in a second position, master processor 20 controls non-compound processor 412 to perform B mode processing, scan conversion, and video processing on one of frames 420 to generate a spatially non-compounded image that is displayed on display monitor 16. When switch 408 is in a third position, master processor 20 controls compound processor 410 and non-compound processor 412 to generate and display the spatially compounded and non-compounded images in display monitor 16.

Additionally, master processor 20 controls color processor 416 to perform color flow processing, to overlay, on display monitor 16, the color flow frames 424 on at least one of the spatially compounded image, and the spatially non-compounded image. Master processor 20 controls timeline processor 414 to perform the M mode processing, scan conversion, and video processing on the M mode data of the timeline frames 422 to generate the M mode image, which is an example of the timeline image, on display monitor 16. Alternatively, timeline processor 414 performs the Doppler processing, scan conversion, and video processing to generate the Doppler image, which is an example of the timeline image, displayed on display monitor 16.

Based on an input provided by the operator via the operator interface 22, master processor 20 controls display monitor 16 to simultaneously display side-by-side at least two of the timeline image, the spatially compounded image, and the spatially non-compounded image. Any of the spatially compounded image and the spatially non-compounded image may be overlaid with the color flow image when displayed side-by-side with another image on display monitor 16. For example, when the operator selects an input on operator interface 22, master processor 20 controls display monitor 16 to simultaneously display the color flow image overlaid over the spatially non-compounded image that is displayed side-by-side with the M mode image. As another example, when the operator selects an input on operator interface 22, master processor 20 controls display monitor 16 to simultaneously display the color flow image overlaid over the spatially compounded image that is displayed side-by-side with the Doppler image.

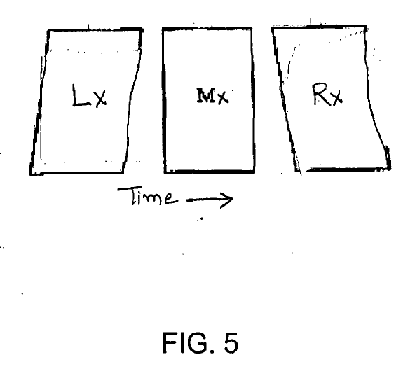

FIG. 5 is an embodiment of a method for acquiring a sequence of frames Lx, Mx, and Rx in real time. Frame Lx is acquired before frame Mx and frame Mx is acquired before frame Rx is acquired. Frames Lx, Mx, and Rx are examples of frames 420 (FIG. 4). Each of frames Lx, Mx, and Rx is formed when master processor 20 (FIG. 1) controls transducer 2 (FIG. 1) to transmit at least one of the transmit firings.

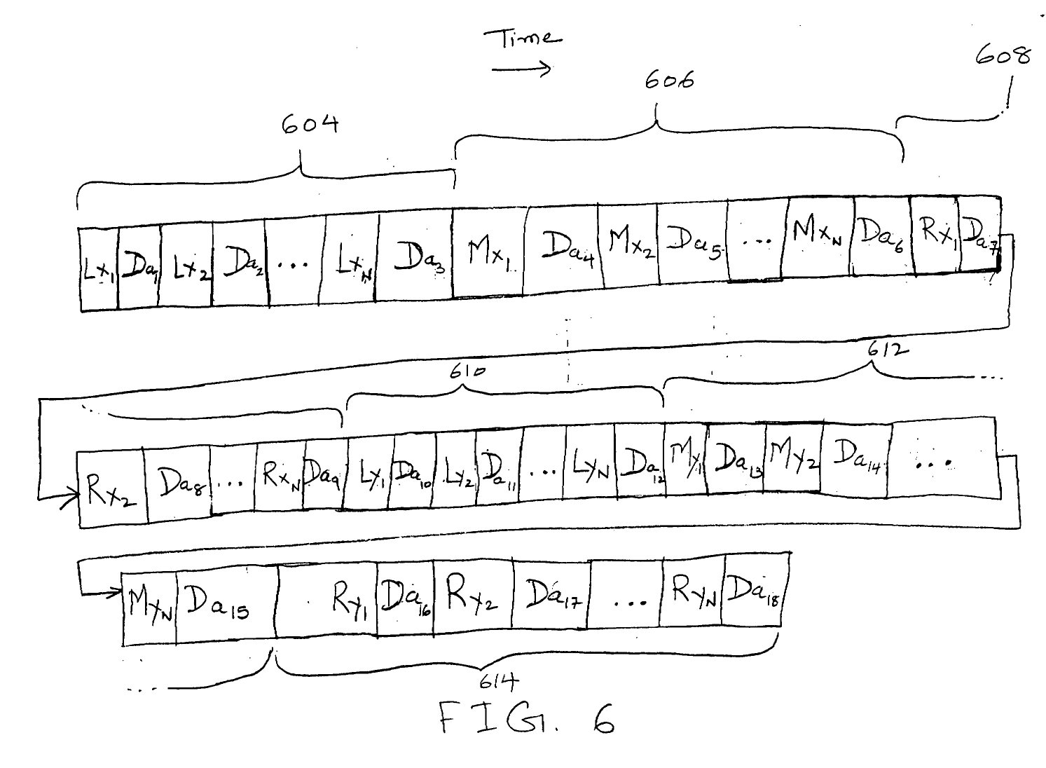

FIG. 6 is an embodiment of a method for acquiring images simultaneously. Master processor 20 (FIG. 1) controls transducer 2 (FIG. 1) to generate the transmit firings from which groups 604, 606, 608, 610, 612, and 614 including subsets Lx₁, Da₁, Lx₂, Da₂, . . . , Lx_{N}, Da₃, Mx₁, Da₄, Mx₂, Da₅, . . . , Mx_{N}, Da₆, Rx₁, Da₇, Rx₂, Da₈, . . . , Rx_{N}, Da₉, Ly₁, Da₁₀, Ly₂, Da₁₁, . . . , Ly_{N}, Da₁₂, My₁, Da₁₃, My₂, Da₁₄, . . . , My_{N}, Da₁₅, Ry₁, Da₁₆, Ry₂, Da₁₇, . . . , Ry_{N}, Da₁₈, are generated as time progresses, where N is an integer representing a number of subsets in a frame.

It is noted that in an alternative embodiment, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, Da₁₈ shown in FIG. 6 are replaced by Da₄, Da₅, Da₆, Da₈, Da₉, Da₁₀, Da₁₂, Da₁₃, Da₁₄, Da₁₆, Da₁₇, Da₁₈, Da₂₀, Da₂₁, Da₂₂, Da₂₄ respectively. In yet another alternative embodiment, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, Da₁₈ shown in FIG. 6 are replaced by Da₅, Da₆, Da₇, Da₁₀, Da₁₁, Da₁₂, Da₁₅, Da₁₆, Da₁₇, Da₂₀, Da₂₁, Da₂₂, Da₂₅, Da₂₆, Da₂₇, Da₃₀ respectively. Any one of Da₂₀, Da₂₁, Da₂₂, Da₂₄, Da₂₅, Da₂₆, Da₂₇, and Da₃₀ is acquired in a similar manner in which Da_{L} is acquired, where L is an integer ranging from 1 to 18.

Each of Lx₁, Lx₂, . . . , Lx_{N} represents a subset of the frame Lx. For example, Lx₁ is formed after master processor 16 controls transducer 2 to transmit at least one of the transmit firings. Similarly, each of Mx₁, Mx₂, . . . , Mx_{N} represents a subset of the frame Mx. For example, Mx₁ is formed after master processor 16 controls transducer 2 to transmit at least one of the transmit firings. Similarly, each of Rx₁, Rx₂, . . . , Rx_{N} represents a subset of the frame Rx. For example, Rx₁ is formed after master processor 16 controls transducer 2 to transmit at least one of the transmit firings.

Each of Ly₁, Ly₂, . . . , Ly_{N} represents a subset of the frame Ly acquired in a similar manner in which Lx is acquired but at a later time than Lx is acquired. Moreover, each of My₁, My₂, . . . , My_{N} represents a subset of the frame My acquired in a similar manner in which Mx is acquired but at a later time than Mx is acquired. Each of Ry₁, Ry₂, . . . , Ry_{N} represents a subset of the frame Ry acquired in a similar manner in which Rx is acquired but at a later time than Rx is acquired.

Each of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ represents at least one subset of the timeline frames 422 (FIG. 4) from which either the Doppler data or alternatively the M mode data is generated. Each of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ is generated when master processor 20 controls transducer 2 to transmit at least one of the transmit firings. For example, each of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ is generated from a number of Doppler firings sufficient to perform at least one FFT and to allow for additional time to make the time between FFTs generated from Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ consistent. The Doppler firings are the transmit firings from which the Doppler data is generated. As another example, each of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₆, Da₁₇, and Da₁₈ is generated from a number of the Doppler firings sufficient to perform an FFT. As yet another example, each of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ is generated from a number of M firings. The M firings are the transmit firings from which the M mode data is generated.

Master processor 20 controls image processors 6 and 8 (FIG. 1), scan converter 12, graphics/timeline display memory 18, video processor 14, display monitor 16, and memory 24 to form images in real time simultaneously with the acquisition of Lx₁, Da₁, Lx₂, Da₂, . . . , Lx_{N}, Da₃, Mx₁, Da₄, Mx₂, Da₅, . . . , Mx_{N}, Da₆, Rx₁, Da₇, Rx₂, Da₈, . . . , Rx_{N}, Da₉, Ly₁, Da₁₀, Ly₂, Da₁₁, . . . , Ly_{N}, Da₁₂, My₁, Da₁₃, My₂, Da₁₄, . . . , My_{N}, Da₁₅, Ry₁, Da₁₆, Ry₂, Da₁₇, . . . , Ry_{N}, Da₁₈. For example, master processor 22 controls display monitor 16 to simultaneously display a portion, generated from Da₁, of the Doppler image with a portion, generated from Lx₂, of the B mode image. As another example, at least two of a set including Lx₁, Lx₂, . . . , Lx_{N}, a set including Mx₁, Mx₂, . . . , Mx_{N}, and a set including Rx₁, Rx₂, . . . , Rx_{N} are spatially compounded to generate the B mode image, which is also the spatially-compounded image. The spatially compounded image is displayed simultaneously with either the Doppler image or the M mode image. Either the Doppler image or the M mode image are generated from at least one of Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈. As yet another example, the spatially non-compounded image is formed from one of the set including Lx₁, Lx₂, . . . , Lx_{N}, set including Mx₁, Mx₂, . . . , Mx_{N}, and set including Rx₁, Rx₂, . . . , Rx_{N}, and displayed simultaneously with either the Doppler image or alternatively the M mode image. As yet another example, the spatially compounded image and the spatially non-compounded image are displayed simultaneously with either the Doppler image or alternatively the M mode image.

Master processor 20 controls transducer 2 (FIG. 1) to interleave the sets including Lx₁, Lx₂, . . . , Lx_{N}, Mx₁, Mx₂, . . . , Mx_{N}, and Rx₁, Rx₂, . . . , Rx_{N} with Da₁, Da₂, Da₃, Da₄, Da₅, Da₆, Da₇, Da₈, Da₉, Da₁₀, Da₁₁, Da₁₂, Da₁₃, Da₁₄, Da₁₅, Da₁₆, Da₁₇, and Da₁₈ as illustrated in FIG. 6. As an example, master processor 20 controls transducer 2 (FIG. 1) to interleave the transmit firings from which Lx₁ and Lx₂ are generated with one of the transmit firings from which Da₁ is generated. As another example, master processor 20 controls transducer 2 (FIG. 1) to interleave the transmit firings from which Rx₁ and Rx₂ are generated with one of the transmit firings from which Da₇ is generated.

When executing the method illustrated in FIG. 5 and upon determining, by master processor 20, that the operator, via the operator interface 22, has selected to apply the method illustrated in FIG. 6, the master processor 20 adjusts at least one parameter for acquiring Lx₁, Lx₂, . . . , Lx_{N}, Mx₁, Mx₂, . . . , Mx_{N}, Rx₁, Rx₂, . . . , Rx_{N}, Ly₂, . . . , Ly_{N}, My₁, My₂, . . . , My_{N}, and Ry₁, Ry₂, . . . , Ry_{N}. Examples of the at least one parameter include performing spatially compounding on at least two of the set including Lx₂, . . . , Lx_{N}, the set including Mx₁, Mx₂, . . . , Mx_{N}, and the set including Rx₁, Rx₂, . . . , Rx_{N}. Other examples of the at least one parameter include a number of the transmit firings fired by transducer 2 to acquire at least one of Lx₁, Lx₂, . . . , Lx_{N}, Mx₁, Mx₂, . . . , Mx_{N}, Rx₁, Rx₂, . . . , and Rx_{N}, and a number of focus points of each of the transmit firings.

When executing the method illustrated FIG. 5 and upon determining, by master processor 20, that the operator has not selected to execute the method of illustrated in FIG. 6, master processor 20 continues to execute the method illustrated in 5. When executing the method illustrated in FIG. 5 and upon determining, by master processor 20, that the operator, via the operator interface 22, has selected to apply the method illustrated in FIG. 6, the master processor 20 changes the at least one parameter to accommodate the method illustrated in FIG. 6. As an example, when executing the method illustrated in FIG. 5 and upon determining, by master processor 20, that the operator, via the operator interface 22, has selected to apply the method illustrated in FIG. 6, the master processor 20 discontinues performing spatial compounding on at least two of the set including Lx₁, . . . , Lx_{N}, the set including Mx₁, Mx₂, . . . , Mx_{N}, and the set including Rx₁, Rx₂, . . . , Rx_{N} and performs non-spatial compounding on one of the sets. As another example, when executing the method illustrated in FIG. 5 and upon determining, by master processor 20, that the operator, via the operator interface 22, has selected to apply the method illustrated in FIG. 6, the master processor 20 reduces a number of the transmit firings used to acquire at least one of Lx₁, Lx₂, . . . , Lx_{N}, Mx₁, Mx₂, . . . , Mx_{N}, Rx₁, Rx₂, . . . , and Rx_{N}. As yet another example, when executing the method illustrated in FIG. 5 and upon determining, by master processor 20, that the operator, via the operator interface 22, has selected to apply the method illustrated in FIG. 6, the master processor 20 reduces a number of focus points along one the transmit firings from which Lx₁ is generated.

When executing the method illustrated in FIG. 5 and upon determination, by the operator, to execute the method illustrated in FIG. 6, the operator provides an operator input to change the at least one parameter to accommodate the execution of the method illustrated in FIG. 6. As an example, when executing the method illustrated in FIG. 5 and upon determining, by the operator, to execute the method illustrated in FIG. 6, the operator controls master processor 20 to discontinue performing spatial compounding on at least two of the set including Lx₁, . . . , Lx_{N}, the set including Mx₁, Mx₂, . . . , Mx_{N}, and the set including Rx₁, Rx₂, . . . , Rx_{N}. As another example, when executing the method illustrated in FIG. 5 and upon determining, by the operator, to execute the method illustrated in FIG. 6, the operator controls master processor 20 to reduce a number of the transmit firings used to acquire at least one of Lx₁, Lx₂, . . . , Lx_{N}, Mx₁, Mx₂, . . . , Mx_{N}, Rx₁, Rx₂, . . . , and Rx_{N}.

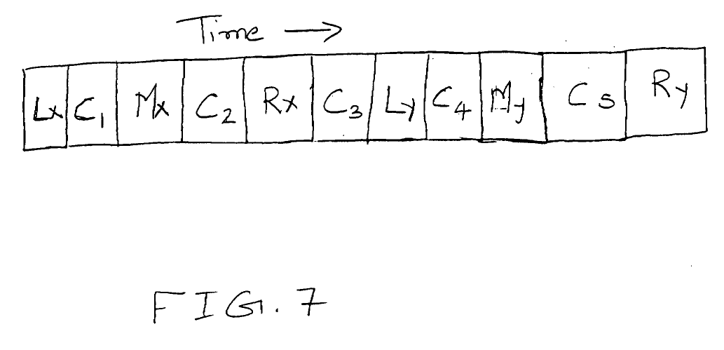

FIG. 7 is an alternative embodiment of a method for acquiring images simultaneously. C₁, C₂, C₃, C₄, and C₅ are examples of color flow frames 424 (FIG. 4). Each of C₁, C₂, C₃, C₄, and C₅ represents a single color flow frame. Master processor 20 controls transducer 2 to generate, as time progresses, the transmit firings from which frames Lx, C₁, Mx, C₂, Rx, C₃, Ly, C₄, My, C₅, and Ry are generated. Master processor 20 controls image processor 6, scan converter 12, video processor 14, memory 24, and display monitor 16 to simultaneously display the B mode image formed from at least one of Lx, Mx, Rx, Ly, My, and Ry with a color flow image formed from at least one of frames C₁, C₂, C₃, C₄, and C₅. The color flow image is simultaneously displayed with and overlaid on the B mode image. As an example, the color flow image is simultaneously displayed with and overlaid over the spatially compounded image formed by combining at least two of frames Lx, Mx, and Rx. As another example, the color flow image is overlaid over and simultaneously displayed with the spatially non-compounded image formed from one of frames Lx, Mx, and Rx.

Master processor 20 controls transducer 2 to interleave Lx, Mx, Rx, Ly, My, and Ry with C₁, C₂, C₃, C₄, and C₅. As an example, master processor 20 controls transducer 2 to interleave one of the transmit firings from which C₁ is generated with the transducer firings from which Lx and Mx are generated. As another example, master processor 20 controls transducer 2 to interleave one of the transmit firings from which My is generated with the transmit firings from which C₄, and C₅ are generated.

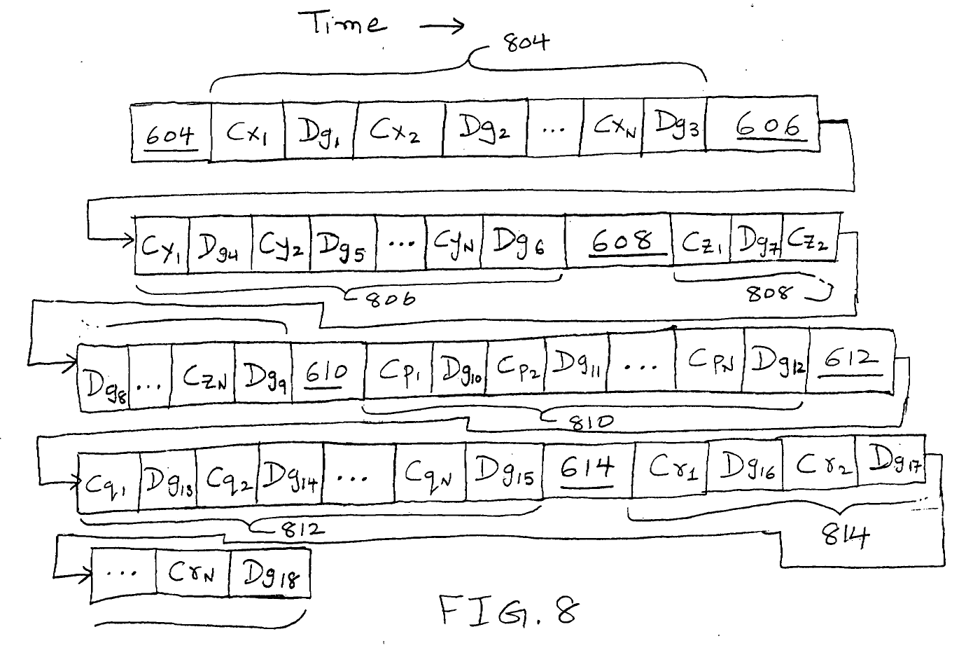

FIG. 8 is an embodiment of a method for acquiring images simultaneously. Master processor 20 controls transducer 2 to generate the transmit firings from which groups 604, 804, 606, 806, 608, 808, 610, 810, 612, 812, 614, and 814 including subsets Lx₁, Da₁, Lx₂, Da₂, . . . , Lx_{N}, Da₃, Cx₁, Dg₁, Cx₂, Dg₂, . . . , Cx_{N}, Dg₃, Mx₁, Da₄, Mx₂, Da₅, . . . , Mx_{N}, Da₆, Cy₁, Dg₄, Cy₂, Dg₅, . . . , Cy_{N}, Dg₆, Rx₁, Da₇, Rx₂, Da₈, . . . , Rx_{N}, Da₉, Cz₁, Dg₇, Cz₂, Dg₈, . . . , Cz_{N}, Dg₉, Ly₁, Da₁₀, Ly₂, Da₁₁, . . . , Ly_{N}, Da₁₂, Cp₁, Dg₁₀, Cp₂, Dg₁₁, . . . Cp_{N}, Dg₁₂, My₁, Da₁₃, My₂, Da₁₄, . . . , My_{N}, Da₁₅, Cq₁, Dg₁₃, Cq₂, Da₁₄, . . . , Cq_{N}, Dg₁₅, Ry₁, Da₁₆, Ry₂, Da₁₇, . . . , Ry_{N}, Da₁₈, Cr₁, Dg₁₆, Cr₂, Da₁₇, . . . , Cr_{N}, Da₁₈. Each of Cx₁, Cx₂, . . . , Cx_{N} represents a subset of frame C₁ (FIG. 7), each of Cy₁, Cy₂, . . . , Cy_{N} represents a subset of frame C₂ (FIG. 7), and each of Cz₁, Cz₂, . . . , Cz_{N} represents a subset of frame C₃ (FIG. 7). Each of Cp₁, Cp₂, . . . Cp_{N} represents a subset of frame C4, each of Cq₁, Cq₂, . . . , Cq_{N} represents a subset of frame C₅ (FIG. 7), and each of Cr₁, Cr₂, . . . , Cr_{N} represents a subset of one of the color flow frames 424 (FIG. 4).

It is noted that in an alternative embodiment, Dg₃, Dg₄, Dg₅, Dg₆, Dg₇, Dg₈, Dg₉, Dg₁₀, Dg₁₁, Da₁₂, Da₁₃, Da₁₄, Dg₁₅, Da₁₆, Dg₁₇, Da₁₈ shown in FIG. 6 are replaced by Dg₄, Dg₅, Dg₆, Dg₈, Dg₉, Dg₁₀, Da₁₂, Dg₁₃, Da₁₄, Dg₁₆, Dg₁₇, Da₁₈, Dg₂₀, Dg₂₁, Dg₂₂, Dg₂₄ respectively. In yet another alternative embodiment, Dg₃, Dg₄, Dag₅, Dg₆, Dg₇, Dg₈, Dg₉, Dg₁₀, Dg₁₁, Da₁₂, Da₁₃, Da₁₄, Da₁₅, Da₁₆, Dg₁₇, Da₁₈ shown in FIG. 6 are replaced by Dg₅, Dg₆, Dg₇, Dg₁₀, Dg₁₁, Da₁₂, Da₁₅, Da₁₆, Da₁₇, Dg₂₀, Dg₂₁, Dg₂₂, Dg₂₅, Dg₂₆, Dg₂₇, Dg₃₀ respectively. Any one of Dg₂₀, Dg₂₁, Dg₂₂, Dg₂₄, Dg₂₅, Dg₂₆, Dg₂₇, and Dg₃₀ is acquired in a similar manner in which Dg_{L} is acquired.

Each of Dg₁, Dg₂, Dg₃, Dg₄, Dg₅, Dg₆, Dg₇, Dg₈, Dg₉, Dg₁₀, Dg₁₁, Da₁₂, Dg₁₃, Da₁₄, Da₁₅, Da₁₆, Da₁₇, and Da₁₈ represent at least one of the transmit firings from which either the Doppler data or the M mode data is generated. Each of Dg₁, Dg₂, Dg₃, Dg₄, Dg₅, Dg₆, Dg₇, Dg₈, Dg₉, Dg₁₀, Dg₁₁, Da₁₂, Dg₁₃, Dg₁₄, Dg₁₅, Da₁₆, Dg₁₇, and Dg₁₈ is generated when master processor 20 controls transducer 2 to transmit at least one of the transmit firings. For example, each of Dg₁, Dg₂, Dg₃, Dg₄, Dg₅, Dg₆, Dg₇, Dg₈, Dg₉, Dg₁₀, Dg₁₁, Da₁₂, Dg₁₃, Dg₁₄, Dg₁₅, Dg₁₆, Dg₁₇, and Da₁₈ is generated from a number of the Doppler firings sufficient to perform at least one FFT and to allow additional time to make the time between FFTs generated from Da₁, Da₂, Da₃, Dg₁, Dg₂, Dg₃, Da₄, Da₅, Da₆, Dg₄, Dg₅, Dg₆, Da₇, Da₈, Da₉, Dg₇, Dg₈, Dg₉, Da₁₀, Da₁₁, Da₁₂, Dg₁₀, Dg₁₁, Da₁₂, Da₁₃, Da₁₄, Da₁₅, Da₁₃, Da₁₄, Dg₁₅, Da₁₆, Da₁₇, Da₁₈, Da₁₆, Da₁₇, and Da₁₈ consistent. As another example, each of Dg₁, Dg₂, Dg₃, Dg₄, Dg₅, Dg₆, Dg₇, Dg₈, Dg₉, Dg₁₀, Dg₁₁, Dg₁₂, Dg₁₃, Dg₁₄, Dg₁₅, Da₁₆, Dg₁₇, and Da₁₈ is generated from a number of the Doppler firings sufficient to perform an FFT. As yet another example, each of Dg₁, Dg₂, Dg₃, Dg₄, Dg₅, Dg₆, Dg₇, Dg₈, Dg₉, Dg₁₀, Dg₁₁, Da₁₂, Dg₁₃, Da₁₄, Da₁₅, Da₁₆, Dg₁₇, and Da₁₈ is generated from a number of the M firings.

Master processor 20 controls image processors 6 and 8 (FIG. 1), scan converter 12, graphics/timeline display memory 18, video processor 14, display monitor 16, and memory 24 to form images in real time simultaneously with the acquisition of Lx₁, Da₁, Lx₂, Da₂, . . . , Lx_{N}, Da₃, Cx₁, Dg₁, Cx₂, Dg₂, . . . , Cx_{N}, Dg₃, Mx₁, Da₄, Mx₂, Da₅, . . . , Mx_{N}, Da₆, Cy₁, Dg₄, Cy₂, Dg₅, . . . , Cy_{N}, Dg₆, Rx₁, Da₇, Rx₂, Da₈, . . . , Rx_{N}, Da₉, Cz₁, Dg₇, Cz₂, Dg₈, . . . , Cz_{N}, Dg₉, Ly₁, Da₁₀, Ly₂, Da₁, . . . , Ly_{N}, Da₁₂, Cp₁, Dg₁₀, Cp₂, Dg₁₁, . . . , Cp_{N}, Da₁₂, My₁, Da₁₃, My₂, Da₁₄, . . . , My_{N}, Da₁₅, Cq₁, Dg₁₃, Cq₂, Da₁₄, . . . , Cq_{N}, Dg₁₅, Ry₁, Da₁₆, Ry₂, Da₁₇, . . . , Ry_{N}, Da₁₈, Cr₁, Da₁₆, Cr₂, Dg₁₇, . . . , Cr_{N}, Dg₁₈. For example, master processor 20 controls display monitor 16 to display a portion, generated from Cx₁, Cx₂, . . . , Cx_{N}, of the color flow image overlaid over the spatially compounded image generated from at least two of the set including Lx₁, Lx₂, . . . , Lx_{N}, the set including Mx₁, Mx₂, . . . , Mx_{N}, and the set including Rx₁, Rx₂, . . . , Rx_{N} simultaneously with a portion, generated from Da₁, Da₂, . . . , Da₃, Dg₁, Dg₂, . . . , Dg₃, of either the M mode image or the Doppler image. As another example, master processor 20 controls display monitor 16 to display a portion, generated from Da₁, Da₂, . . . , Da₃, Dg₁, Dg₂, . . . , Dg₃, of either the M mode image or the Doppler image with the spatially compounded image generated from at least two of the set including Lx₁, Lx₂, . . . , Lx_{N}, the set including Mx₁, Mx₂, . . . , Mx_{N}, and the set including Rx₁, Rx₂, . . . , Rx_{N}.

As yet another example, master processor 20 controls display monitor 16 to display a portion, generated from Cx₁, Cx₂, . . . , Cx_{N}, of the color flow image overlaid over the spatially non-compounded image generated from one of the set including Lx₁, Lx₂, . . . , Lx_{N}, the set including Mx₁, Mx₂, . . . , Mx_{N}, and the set including Rx₁, Rx₂, . . . , Rx_{N} simultaneously with a portion, generated from Da₁, Da₂, . . . , Da₃, Dg₁, Dg₂, . . . , Dg₃, of either the M mode image or the Doppler image. As another example, master processor 20 controls display monitor 16 to display a portion, generated from Da₁, Da₂, . . . , Da₃, Dg₁, Dg₂, . . . , Dg₃, of either the M mode image or the Doppler image with the spatially non-compounded image generated from one of the set including Lx₁, Lx₂, . . . , Lx_{N}, the set including Mx₁, Mx₂, . . . , Mx_{N}, and the set including Rx₁, Rx₂, . . . , Rx_{N}.

When executing either the method illustrated in FIG. 6 or the method illustrated in FIG. 7 and upon determining, by master processor 20, that the operator, via the operator interface 22, has selected to apply the method illustrated in FIG. 8, the master processor 20 adjusts the at least one parameter for acquiring Lx₁, Lx₂, . . . , Lx_{N}, Mx₁, Mx₂, . . . , Mx_{N}, Rx₁, Rx₂, . . . , Rx_{N}, Ly₂, . . . , Ly_{N}, My₁, My₂, . . . , My_{N}, and Ry₁, Ry₂, . . . , Ry_{N}. Additional examples of the at least one parameter include a number of the transmit firings fired by transducer 2 to acquire at least one of Cx₁, Cx₂, . . . , Cx_{N}, Cy₁, Cy₂, . . . , Cy_{N}, Cz₁, Cz₂, . . . , Cz_{N}, Cp₁, Cp₂, . . . , Cp_{N}, Cq₁, Cq₂, . . . , Cq_{N}, Cr₁, Cr₂, . . . , Cr_{N} and a number of focus points of each of the transmit firings. The additional examples of the at least one parameter are applicable when a determination is made to switch from executing either the method illustrated in FIG. 6 or FIG. 7 to the method illustrated in FIG. 8.

When executing either the method illustrated in FIG. 6 or the method illustrated in FIG. 7 and upon determining, by master processor 20, that the operator has not selected to execute the method of illustrated in FIG. 8, master processor 20 continues to execute one of the methods illustrated in FIG. 6 and FIG. 7 that is currently being executed. When executing either the method illustrated in FIG. 6 or the method illustrated in FIG. 7 and upon determining, by master processor 20, that the operator, via the operator interface 22, has selected to apply the method illustrated in FIG. 8, the master processor 20 changes the at least one parameter to accommodate the method illustrated in FIG. 8. As an example, when executing either the method illustrated in FIG. 6 or the method illustrated in FIG. 7 and upon determining, by master processor 20, that the operator, via the operator interface 22, has selected to apply the method illustrated in FIG. 8, the master processor 20 discontinues performing spatial compounding on at least two of the set including Lx₁, . . . , Lx_{N}, the set including Mx₁, Mx₂, . . . , Mx_{N}, and the set including Rx₁, Rx₂, . . . , Rx_{N} and performs non-spatial compounding on one of the sets. As another example, when executing either the method illustrated in FIG. 6 or the method illustrated in FIG. 7 and upon determining, by master processor 20, that the operator, via the operator interface 22, has selected to apply the method illustrated in FIG. 8, the master processor 20 reduces a number of the transmit firings used to acquire at least one of Lx₁, Lx₂, . . . , Lx_{N}, Mx₁, Mx₂, . . . , Mx_{N}, Rx₁, Rx₂, . . . , Rx_{N}, Cx₁, Cx₂, . . . , Cx_{N}, Cy₁, Cy₂, . . . , Cy_{N}, Cz₁, Cz₂, . . . , Cz_{N}, Cp₁, Cp₂, . . . , Cp_{N}, Cq₁, Cq₂, . . . , Cq_{N}, Cr₁, Cr₂, . . . , and Cr_{N}.

When executing either the method illustrated in FIG. 6 or the method illustrated in FIG. 7 and upon determination, by the operator, to execute the method illustrated in FIG. 8, the operator provides an operator input to change the at least one parameter to accommodate the execution of the method illustrated in FIG. 8. As an example, when executing either the method illustrated in FIG. 6 or the method illustrated in FIG. 7 and upon determining, by the operator, to execute the method illustrated in FIG. 8, the operator controls master processor 20 to discontinue performing spatial compounding on at least two of the set including Lx₂, . . . , Lx_{N}, the set including Mx₁, Mx₂, . . . , Mx_{N}, and the set including Rx₁, Rx₂, . . . , Rx_{N} and to perform non-spatial compounding on one of the sets. As another example, when executing either the method illustrated in FIG. 6 or the method illustrated in FIG. 7 and upon determining, by the operator, to execute the method illustrated in FIG. 8, the operator controls master processor 20 to reduce a number of the transmit firings used to acquire at least one of Lx₁, Lx₂, . . . , Lx_{N}, Mx₁, Mx₂, . . . , Mx_{N}, Rx₁, Rx₂, . . . , Rx_{N}, Cx₁, Cx₂, . . . , Cx_{N}, Cy₁, Cy₂, . . . , Cy_{N}, Cz₁, Cz₂, . . . , Cz_{N}, Cp₁, Cp₂, . . . , Cp_{N}, Cq₁, Cq₂, . . . , Cq_{N}, Cr₁, Cr₂, . . . , and Cr_{N}.

Master processor 20 controls transducer 2 to interleave groups 804, 806, 808, 810, 812, and 814 with groups 604, 606, 608, 610, 612, and 614. For example, master processor 20 controls transducer 2 to interleave the transmit firings from which group 804 is generated with the transmit firings from which groups 604 and 606 are generated. Master controller 20 controls transducer 2 to interleave Dg₁, Dg₂, . . . , Dg₃ with Cx₁, Cx₂, . . . , Cx_{N}, to interleave Dg₄, Dg₅, . . . , Dg₆ with Cy₁, Cy₂, . . . , Cy_{N}, to interleave Cz₁, Cz₂, . . . , Cz_{N} with Dg₇, Dg₈, . . . , Dg₉, to interleave Cp₁, Cp₂, . . . , Cp_{N} with Dg₁₀, Dg₁₁, . . . , Da₁₂, to interleave Cq₁, Cq₂, . . . , Cq_{N} with Da₁₃, Da₁₄, . . . , Da₁₅, to interleave Cr₁, Cr₂, . . . , Cr_{N} with Dg₁₆, Dg₁₇, . . . , Dg₁₈. For example, master processor 20 controls transducer 2 to interleave at least one of the transmit firings from which Dg₁ is generated with the transmit firings from which Cx₁ and Cx₂ are generated.

Technical effects of the systems and methods for acquiring images simultaneously include simultaneously acquiring at least one of the spatially compounded image of the sample volume and the spatially non-compounded image of the sample volume with either the M mode image of the sample volume or the Doppler image of the sample volume. The operator is productive in making a diagnosis by simultaneously viewing at least one of the spatially compounded image and the spatially non-compounded image with either the M mode image or the Doppler image. Anatomical image quality improvements are provided by spatially compounding simultaneous with receipt of physiological information obtained from either the M mode image or the Doppler image. Moreover, other technical effects of the systems and methods for acquiring images simultaneously include changing at least one parameter based on a selection to execute the method illustrated in either FIG. 8 or FIG. 6. The change in the at least one parameter accommodates simultaneous viewing of at least one of the spatially compounded image and the spatially non-compounded image with either the M mode image or the Doppler image.

While the invention has been described in terms of various specific embodiments, those skilled in the art will recognize that the invention can be practiced with modification within the spirit and scope of the claims.

What is claimed is:

1. A method for acquiring images simultaneously, said method comprising simultaneously acquiring a first image with a second image, wherein the first image includes at least one of a spatially compounded image and a spatially non-compounded image, and the second image includes one of an M mode image and a Doppler image.

2. A method in accordance with claim 1 wherein said simultaneously acquiring comprises simultaneously acquiring the first image with the second image and a color flow image.

3. A method in accordance with claim 1 wherein said simultaneously acquiring comprises acquiring the Doppler image by one of continuously transmitting a series of pulses toward a subject and periodically transmitting at least two of the series of pulses toward the subject.

4. A method in accordance with claim 1 further comprising displaying the first image and the second image on a screen of a display device.

5. A method in accordance with claim 1 wherein said simultaneously acquiring comprises simultaneously acquiring the first image with the second image and a color flow image and said method further comprising displaying the first image, the second image, and the color flow image on a screen of a display device.

6. A method in accordance with claim 1 wherein said simultaneously acquiring comprises:
acquiring the first image by transmitting at least one B mode pulse; and
acquiring the second image by transmitting at least one Doppler pulse interleaved with the at least one B mode pulse.

7. A method in accordance with claim 1 wherein said simultaneously acquiring comprises:
acquiring the first image by transmitting at least one B mode pulse; and
acquiring the second image by transmitting at least one M mode pulse interleaved with the at least one B mode pulse.

8. A method in accordance with claim 1 wherein said simultaneously acquiring comprises simultaneously acquiring the first image with the second image and a color flow image by:
acquiring the first image by transmitting at least one B mode pulse;
acquiring the second image by transmitting at least one Doppler pulse interleaved with the B mode pulse; and
acquiring the color flow image by transmitting at least one color flow pulse interleaved with the at least one B mode pulse and the at least one Doppler pulse.

9. A method in accordance with claim 1 wherein said simultaneously acquiring comprises simultaneously acquiring the first image with the second image and a color flow image by:
acquiring the first image by transmitting at least one B mode pulse;
acquiring the second image by transmitting at least one M mode pulse interleaved with the B mode pulse; and
acquiring the color flow image by transmitting at least one color flow pulse interleaved with the at least one B mode pulse and the at least one M mode pulse.

10. A method in accordance with claim 1 further comprising:
automatically determining, without operator intervention, whether at least one parameter for acquiring the second image is changed during said simultaneous acquisition; and
automatically, without operator intervention, changing at least one parameter for acquiring the first image upon determining that the at least one parameter for acquiring the second image is changed.

11. A method in accordance with claim 1 further comprising:
determining, without operator intervention, whether at least one parameter for acquiring the second image is changed during said simultaneous acquisition; and
automatically, without operator intervention, changing at least one parameter for acquiring the first image upon determining that the at least one parameter for acquiring the second image is changed, wherein the at least one parameter for acquiring the first image comprises at least one of a number of at least one pulse transmitted to acquire the first image, a number of two-dimensional images acquired and compounded to form the first image, and a number of focus points of the at least one pulse transmitted to acquire the first image.

12. A method in accordance with claim 1 further comprising:
manually determining whether at least one parameter for acquiring the second image is changed during said simultaneous acquisition; and
manually changing at least one parameter for acquiring the first image upon determining that the at least one parameter for acquiring the second image is changed.

13. A method in accordance with claim 1 further comprising:
determining, without operator intervention, whether said simultaneously acquiring comprises simultaneously acquiring the first image with the second image and a color flow image; and
automatically, without operator intervention, changing at least one parameter for acquiring the first image upon determining that the color flow image is simultaneously acquired with the first image and the second image.

14. A method in accordance with claim 1 further comprising:
manually determining whether said simultaneously acquiring comprises simultaneously acquiring the first image with the second image and a color flow image; and
manually changing at least one parameter for acquiring the first image upon determining that the color flow image is simultaneously acquired with the first image and the second image.

15. A processor configured to control a simultaneous acquisition of a first image with a second image, wherein the first image includes at least one of a spatially compounded image and a spatially non-compounded image, and the second image includes one of an M mode image and a Doppler image.

16. A processor in accordance with claim 15 configured to control the simultaneous acquisition of the first image with the second image and a color flow image.

17. A processor in accordance with claim 15 configured to:
control an acquisition of the first image by controlling a transmission of at least one B mode pulse; and
control an acquisition of the second image by controlling a transmission of at least one Doppler pulse interleaved with the at least one B mode pulse.

18. An ultrasound imaging system comprising:
a plurality of transducer elements configured to receive a plurality of ultrasound echoes and convert the ultrasound echoes to a plurality of electrical signals;
a beamformer board coupled to said transducer elements and configured to generate a receive beam from the electrical signals;
a first image processor coupled to said beamformer and configured to generate a first image output from the receive beam;
a second image processor coupled to said beamformer and configured to generate a second image output from the receive beam; and
a master processor configured to control said transducer elements, said beamformer, said first image processor, and said second image processor to simultaneously acquire a first image formed from the first image output with a second image formed from the second image output, wherein the first image includes at least one of a spatially compounded image and a spatially non-compounded image, and the second image includes one of an M mode image and a Doppler image.

19. An ultrasound imaging system in accordance with claim 18 further comprising a color flow image processor coupled to said beamformer and configured to generate a color flow image output from the receive beam, wherein said master processor is configured to control said transducer elements, said beamformer, said first image processor, said second image processor, and said color flow image processor to simultaneously acquire a first image formed from the first image output, a second image formed from the second image output, and a color flow image from the color flow image output.

20. An ultrasound imaging system in accordance with claim 18 wherein said master processor is configured to control the simultaneously acquisition of the first image with the second image by controlling a transmission of at least one B mode pulse and by controlling a transmission of at least one Doppler pulse to interleave with the at least one B mode pulse.
